(19) United States
(12) Patent Application Publication
(10) Pub. No.: US 2016/0279069 A1
(43) Pub. Date: Sep. 29, 2016

(54) MATERIALS AND METHODS FOR SUSTAINED RELEASE OF ACTIVE COMPOUNDS

(71) Applicants: Rahul Dev JAYANT, Miami, FL (US); Madhavan NAIR, Coral Gables, FL (US)

(72) Inventors: Rahul Dev JAYANT, Miami, FL (US); Madhavan NAIR, Coral Gables, FL (US)

(73) Assignee: The Florida International University Board of Trustees, Miami, FL (US)

(21) Appl. No.: 15/082,611

(22) Filed: Mar. 28, 2016

Related U.S. Application Data
(60) Provisional application No. 62/138,797, filed on Mar. 26, 2015.

(51) Int. Cl. A61K 9/50; A61K 9/127; A61K 31/16; A61K 9/51; A61K 31/675

(52) U.S. Cl. CPC A61K 9/5078; A61K 9/5094; A61K 9/5161; A61K 31/675; A61K 31/16; A61K 9/127

(57) ABSTRACT

The subject invention provides nanoparticle drug delivery systems and methods of making and using the same. In one aspect, the present invention provides a nanoparticle drug delivery system comprising a magnetic nanoparticle (MNP) encapsulated by at least one bilayer coating comprising a layer of drug molecules and a layer of polymer. In another aspect, a method of using the nanoparticle drug delivery system can include: administering the nanoparticle drug delivery system systemically and localizing the nanoparticles to the target treatment area. In embodiments the nanoparticle drug delivery system is used to treat HIV-AIDS.

### CROSS-REFERENCE TO RELATED APPLICATIONS

This application claims the priority benefit of U.S. Provisional Application Ser. No. 62/138,797, filed Mar. 26, 2015 which is incorporated herein by reference in its entirety.

### GOVERNMENT SUPPORT

This invention was made with government support under grant numbers 1RO1DA027049, 1R21MH101025, RO1-DA034547, RO1-DA037838 and RO1-DA040537 awarded by the National Institutes of Health. The government has certain rights in the invention.

### BACKGROUND

Subsequent to the introduction of combination antiretroviral therapy (ART), HIV-infection-related morbidity and mortality have dramatically decreased; however, currently available antiretroviral agents, such as those involved in highly active antiretroviral therapy (HAART), are only capable of controlling HIV replication rather than completely eradicating virus from patients. As a result, HIV infection has now become a chronic disease requiring a lifelong commitment to daily oral treatment.

HAART comprises complex regimens that require strict adherence to complicated treatment schedules, and the quality of treatment depends on the patient's adherence to the recommended regimens. Antiretroviral adherence is the second strongest predictor of progression to AIDS and death, after CD4 count (Garcia de Olalla, P. et al., J Acquir Immune Defic Syndr. 2002; 30(1):105-110; Nowacek, A. et al., Nanomedicine (Lond). 2009; 4(5):557-574).

Most antiretroviral drugs have a short half-life and, in turn, need to be in circulation constantly to control the virus replication. As a result, it is believed that missing a medication dose even once can provide an opportunity for viruses to replicate such that a medication-resistant HIV strain may develop. Long-acting formulations of therapeutic agents have been used to improve adherence and prevent issues such as missing doses or treatment fatigue to prescribed medication in a number of different fields such as contraception, male hypogonadism, and schizophrenia, with demonstrable success (Boffito, M. et al., Drugs. 2014; 74(1):7-13). Thus, it has been suggested that development of similar approaches in reducing the impact of individual adherence could increase the efficacy of treatment strategies for HIV-AIDS.

On the other hand, current HAART only helps in suppressing HIV replication and does not clear virus from infected individuals. Various reservoirs of replication-competent HIV have been identified that may contribute to this persistence. One example of such a reservoir is the brain, which serves as one of the sources of virus production to the peripheral nervous system. This remains a formidable challenge due to the inability of conventional ART to penetrate the blood-brain barrier and subsequently deprive the brain from the drug effects (Thomas, S. A. et al., Curr Pharm Des. 2004; 10(12):1313-1324; Ayre, S. G. et al., Med Hypotheses. 1989; 29(4):283-291; Bhaskar, S. et al., Part Fibre Toxicol. 2010; 7(1):3; Pardridge, W. M. et al., Drug Discov Today. 2007; 12(1-2):54-61; Sagar, V. et al., Rev Med Virol. 2014; 24(2):103-124; Saiyed, Z. M. et al., Int J Nanomedicine. 2010; 5:157-166; Varatharajan, L. et al., Antiviral Res. 2009; 82(2):A99-A109).

### BRIEF SUMMARY

The present invention provides novel nanoparticle drug delivery systems and methods of making and using the same.

The present invention provides a nanoparticle drug delivery system comprising a magnetic nanoparticle (MNP) encapsulated by at least one bilayer coating comprising a layer of drug molecules and a layer of polymer. In some embodiments, there can be more than one bilayer coating around the MNP, with each bilayer comprising a layer of drug molecules and a layer of a polymer, where the drug molecules can be the same as, or distinct from, the first drug molecules, and the polymer can be the same as, or distinct from, the first polymer.

In one embodiment, the subject invention provides a system that facilitates directed targeting of drugs to an area within an animal body, preferably a human body, whereby the targeted area can be, for example, an organ or a site within (or on the surface of) the animal (e.g., human) body. Examples of such areas include the blood brain barrier (BBB) and the brain.

In certain embodiments, the MNP comprises iron oxide. In an exemplary embodiment, the MNP comprises Fe₃O₄ and/or Fe₂O₃.

In certain embodiments, the drug and the polymer of a bilayer are oppositely charged polyanion and polycation. In specific embodiments, the polymer is negatively charged. In other embodiments, the polymer is positively charged. In exemplary embodiments the bilayer structure is assembled such that the oppositely charged drug molecules and polymer adsorb onto the MNP in an alternating, layer-by-layer (LbL) fashion.

Advantageously, embodiments of the nanoparticle drug delivery system can be prepared by methods that are more simple than methods of manufacturing drug targeting systems known from the prior art.

Further, in accordance with the present invention, a nanoparticle drug delivery system can be delivered to a mammal more conveniently, e.g., by a simple administration step and can be directed such as by magnetic force to the desired target in a high rate and with a high ratio of transfer of the drug from the site of administration to the site of pharmacological effect.

In certain embodiments, the release of the drug can be sustained for a longer period of time than when using nanoparticles comprising only one drug without the bilayer coating.

Certain embodiments of the subject invention directed to treating viral diseases such as HIV-AIDS provide that the drug in one bilayer coating is an antiretroviral drug and the drug in a second layer is a viral latency-activating drug. In an exemplary embodiment, the drug in one bilayer coating may be tenofovir (teno), a nucleoside reverse transcriptase inhibitor (NRTI), and the drug in another layer may be vorinostat (vor), a histone deacetylase (HDAC) inhibitor.

In specific embodiments the invention provides a method of delivering pharmacologically active substances across the blood brain barrier (BBB). Thus, in an exemplary embodiment the treatment is for a viral infection, which may be latent or active, and the area may be the brain. Advantageously, the nanoparticle drug delivery system of the subject invention has the ability to cross the BBB in order to treat the infected tissue.

In one embodiment, the method of the invention comprises the steps of preparing the nanoparticle drug delivery system with at least one drug, administering the nanoparticles to an animal in a manner that allows the drug to reach and cross the BBB, and allowing the drug to be released from the nanoparticle to achieve the desired pharmacological effect in a sustained manner.

In yet another embodiment, the subject drug delivery system can be used as an antagonist to substances of abuse, including, but not limited to, cocaine, morphine, and methamphetamine, and reverse the neurodegenerative effects of such substances.

### BRIEF DESCRIPTION OF THE DRAWINGS

FIG. 1 is a schematic representation of the structure of a nanoparticle drug delivery system for treating HIV-AIDS and the method of delivery controlled by an external magnetic field. The infected tissue in this exemplary illustration is located in the human brain and the target delivery involves mobilizing the drug delivery system across the blood-brain barrier (BBB).

FIG. 2 is a schematic representation of an exemplary embodiment of the drug delivery system with the formulation design {MNP+(tenofovir+dextran sulfate)₂+vorinostat}.

FIG. 3A is a transmission electron microscopy (TEM) image of uncoated magnetic nanoparticles (MNPs).

FIG. 3B is a TEM image of MNPs coated with (tenofovir+dextran sulfate)₂+vorinostat.

FIG. 4 is a schematic representation of the layer-by-layer (LbL) assembly process.

FIG. 5 illustrates an exemplary zeta-potential analysis for the confirmation of LbL deposition on MNPs.

FIG. 6 demonstrates the effect of dextran sulphate sodium layer coating on the tenofovir loading profile in one embodiment of the drug delivery system of the subject invention.

FIG. 7 compares the in-vitro cumulative pharmacokinetic release data of uncoated tenofovir, uncoated vorinostat, and dextran-coated (1 bilayer [1BL] and two bilayers [2BL]) drug delivery systems in phosphate-buffered saline (pH, 7.4) at approximately 37° C. Inset shows the initial burst release profile at 4 hours for all the nanoformulations.

FIG. 8 depicts the results of an exemplary p24 study demonstrating that the magnetically triggered release does not affect the inhibition efficacy of tenofovir in HIV replication. Release drug and standard drug (approximately 1 mg/mL) were used for efficacy assay in human astrocytes. Treatment was done for 24 hours, and results were analyzed via p24 antigen estimation, using enzyme-linked immunosorbent assay on the fifth day of infection and expressed as picograms per milliliter.

FIG. 9 is a schematic representation of an exemplary in-vitro BBB model comprising primary human astrocytes co-cultured with primary human brain microvascular endothelial cells. The culture plate was bi-compartmentalized via a transwell porous membrane. The top and underside of this membrane were respectively cultured with endothelial cells with tight junctions and astrocytes, which correspondingly mimics the external (peripheral blood side) and internal (brain microenvironment side) surface of BBB.

FIG. 10A illustrates the MNP drug delivery system transmigration rate across the exemplary in-vitro BBB model. MNP drug delivery system was added in the upper chamber of the BBB model with a magnet (0.8 T) placed underneath for the duration of experiment. At about 6 hours after plating, migrated uncoated MNPs and MNP drug delivery system were calculated for iron content in the lower chamber. Results are expressed as mean±standard error of three independent experiments. Statistical significance was determined using unpaired Student's t-test. NS indicates when P-value was found to be more than 0.05; then the result would be considered statistically not significant.

FIG. 10B demonstrates the effect of an embodiment of the drug delivery system on fluorescein isothiocyanate (FITC)-dextran transport in the BBB model. FITC-dextran transport was measured after about 6 hours of magnetic treatment. FITC-dextran was added to the upper chamber of the insert. After about 6 hours of incubation, relative fluorescence units from the basal chambers of the inserts were measured. Results are expressed as percentage FITC-dextran transport with respect to the untreated control cultures (negative control=no cell and positive control=with cells) and represented as mean±standard error of independent experiments.

FIG. 11 represents an evaluation of the antiviral efficacy of an exemplary embodiment of the drug delivery system in suppressing p24 antigen levels in HIV-infected human astrocytes. The drug delivery system was added on the second day after HIV infection (HIV-1BaL) in equimolar concentration. Supernatants were evaluated on the third, fifth, and seventh day postinfection for p24 levels and were checked for its efficacy, along with its percentage cell viability, using MTT assay. Simultaneous incorporation of tenofovir and vorinostat into the magnetic nanoparticle nanoformulation leads to latent virus activation and inhibits viral spread almost comparable to tenofovir nanoformulation. Viral p24 protein in the culture supernatant was measured by enzyme-linked immunosorbent assay. Error bars indicate the standard deviation of triplicate data points and are representative of at least three experiments. Dotted lines represent the percentage cell viability; each point represents treatment period. If mean P-value was found to be less than 0.05, then the result would be considered statistically significant. NS=non-significant. MTT=3-(4,5-dimethylthiazol-2-yl)-2,5-diphenyltetrazolium bromide.

FIG. 12 shows the DLS measurement of hydrodynamic diameter of the drug delivery system provided herein with respect to drug loading and LbL assembly deposition.

FIG. 13A shows an exemplary magnetic hysteresis loop of MNPs.

FIG. 13B shows an exemplary X-ray diffraction (XRD) spectrum of MNPs showing magnetic-specific characteristic planes as labeled.

FIG. 14A shows an exemplary dataset for Vorinostat dose optimization for latency reversal in human astrocytes. Different ranges of vorinostat (from about 1 to about 10 μM) were used and tested for p24 value. Results showed that the maximum p24 value at 1-μM concentration, which was employed for an exemplary embodiment of the drug delivery system.

FIG. 14B shows an exemplary dataset of cytotoxicity evaluation of vorinostat dosage using MTT assay in human astrocytes. Different ranges of vorinostat (from about 1 to about 10 μM) were used and tested for cellular toxicity after 24 hrs of treatment.

FIGS. 15A-15C show the number of counts versus the mass-to-charge ratio at the standard peak of 288 of embodiments of the drug delivery system. FIG. 15A is the analysis for the standard free tenofovir (1:10 water dilution). FIG. 15B shows the released tenofovir from a 1-bilayer (1BL) drug delivery system (MNP+teno+DS). FIG. 15C shows the released tenofovior from a 2-bilayer (2BL) drug delivery system {(teno+DS)₂+vor}.

FIGS. 16A-16D depict cellular uptake of the FITC-tagged MNP drug delivery system (concentration=approximately 100 μg/mL) in primary human astrocytes after about 6 hours of treatment. FIG. 16A shows the bright field (BF) image. FIG. 16B shows the image of the cell nuclei stained with DAPI. FIG. 16C shows the distribution of an exemplary drug-loaded 2-layer MNP drug delivery system. FIG. 16D is a composite image of the cell nuclei and the drug delivery system, showing fluorescence inside the cells, and thus confirms the cellular uptake of the drug delivery system. Images were taken at 10× magnification (inset image magnifications, 20×), using fluorescence microscopy (Leica, Wetzlar, Germany). DAPI=4,6-diamidino-2-phenylindole, dihydrochloride.

FIGS. 17A-17D demonstrate a quantitative analysis of FITC-tagged MNP drug delivery system after approximately 24 hours of treatment in primary human astrocytes by flow cytometry. Data suggest that an increase in concentration from about 50 to about 200 μg/mL leads to an increase in the percentage of cells positive for FITC-tagged drug delivery system, as well as an increase in mean fluorescence intensity. Ten thousand events were collected for each sample. Cells were gated on the basis of cells without the drug delivery system (unshaded solid-lined histogram) and the drug delivery system alone (unshaded dotted-lined histogram) controls. Cells positive for the drug delivery system are shown as shaded histograms with shifted mean fluorescence intensity compared with the controls. Shaded histograms overlaid with spectra of the cell control and the MNP drug delivery system alone represent cells treated with approximately 50 μg/mL (FIG. 17A), approximately 100 μg/mL (FIG. 17B), and approximately 200 μg/mL (FIG. 17C) of the FITC-tagged drug delivery system, which shows an approximately 35.7%, approximately 40%, and approximately 44% increase, respectively, in cells positive for the drug delivery system and approximately 59,395, approximately 77,380, and approximately 139,109 mean fluorescence intensity, respectively. In FIG. 17D, the combined histogram shows an overlay of all three concentration levels.

FIG. 18 demonstrates cell viability of human astrocytes with an exemplary embodiment of the MNP drug delivery system. Results show the percentage of cells viable after treatment with different concentrations of the drug delivery system for about 24 hours using MTT assay.

FIG. 19 shows the cell viability of neuronal cells (i.e., SK-N-MC cells) with an exemplary embodiment of the MNP drug delivery system. Results show the percentage of cells after treatment with different concentrations for 24 hours using MTT assay.

FIGS. 20A-20C represent an in vivo cytotoxicity analysis of an exemplary drug delivery system based on illustrative histopathology of the cerebellum (brain) from BALB/C mice treated with approximately 20 mg/kg of 2 bilayer (2BL) MNP+(teno+DS)₂ formulation (FIG. 20C), with only the MNP (FIG. 20B), and about 0.9% saline as the control (FIG. 20A) with about 0.8 Tesla of external magnetic field. Analysis was carried out following approximately 48 hours of treatment. Images were taken after injection and analyses for H & E staining (Scale bar=50 μm).

FIGS. 21A and 21B represent the results of in-vitro phantom studies for detection of MNPs using an MRI machine. T2 (FIG. 21B) and T1 (FIG. 21A) relaxation rates were assessed on a series of phantom tubes filled with a mixture of agarose and varying concentrations of NPs (from about 1 to about 100 μg). The different data series represent phantoms with different intensity. Different T2s were assigned different data series for better visual representation and the confirmation that the MNPs have altered T2 signal intensity.

FIGS. 22A-22C show the results of MRI and image analysis. FIG. 22A shows that the T2 maps of mice brain were reconstructed from a non-linear regression of the exponential decay signal using the multi-TE value datasets. Images were imported into NIH Image J (rsbweb.nih.gov/ij) and the QuickVol plugin1 (http://www.quickvol.com) for processing. FIG. 22B shows the results of mice brain after they were first scanned without any treatment to establish a baseline and then scanned following intravenous administration of an exemplary MNP drug delivery system without any magnetic treatment. The scanning lasted four about 6 hrs with one scan collected about every 30 min. The following parameters were used for T2 relaxation: echo time (TE)=10-200 at 10 ms intervals (20 TE's total), repetition time (TR)=3000 ms, field of view 24 mm² along the read, phase directions and 1 mm along the slice direction, and data matrix of 128×128×10 slices. Parameters similar to those indicated above were used for T2 mapping. T1 was also accessed using a saturation recovery sequence with TR (in ms)=50, 500, 950, 1400, 1850, 2300, 2750, 3200, 3650, 4100, 4550, 5000. FIG. 22C shows the results of the an experiment in which repetition of the above parameters was done, with the exception that in this experiment the mice were injected with the MNP drug delivery system and subjected to about 3 hrs of noninvasive magnetic field treatment and then used for MNP detection using MRI. Regions of interest (ROIs) were manually delineated using the ITK-Snap program. Areas were selected near the ventricles (dorsomedial striatum near lateral ventricles, hypothalamus near the 3^{rd} ventricle) as well as on 3 different ventricular areas (dorsal 3^{rd} ventricle, lateral ventricles, and the cerebral aqueduct).

### DETAILED DESCRIPTION

The present invention provides novel nanoparticle drug delivery systems and methods of making and using the same.

In one aspect, the present invention provides a nanoparticle drug delivery system comprising a magnetic nanoparticle (MNP) encapsulated by at least one bilayer coating comprising a layer of drug molecules and a layer of polymer. In some embodiments, there can be more than one bilayer, with each bilayer additional to the first comprising a layer of drug molecules and a layer of a polymer, where the drug molecules can be the same as, or distinct from, the first drug molecules and the polymer can be the same as, or distinct from, the first polymer.

In some embodiments, the nanoparticle drug delivery system further comprises additional molecules between each layer of drug molecules and polymer. Non-limiting examples of the additional molecules within each bilayer coating include drug molecules, proteins, therapeutic agents, and combinations thereof. Advantageously, the various agents within each bilayer of the drug delivery system help modulate the release profile of the drug molecules in a controlled fashion.

Nanoparticles of the subject invention can include carrier structures that are biocompatible and sufficiently resistant to chemical and/or physical destruction by the environment of use such that a sufficient amount of the nanoparticles remain substantially intact after entry into the animal body following administration so as to be able to reach a desired target area within the mammalian body, e.g., the brain and/or the BBB in the central nervous system (CNS).

In certain embodiments, the drug and the polymer of a bilayer are oppositely charged polyanion and polycation. In specific embodiments, the polymer is negatively charged. In other embodiments, the polymer is positively charged. In exemplary embodiments the bilayer structure is assembled such that the oppositely charged drug molecules and polymer adsorb onto the MNP in an alternating, layer-by-layer (LbL) fashion.

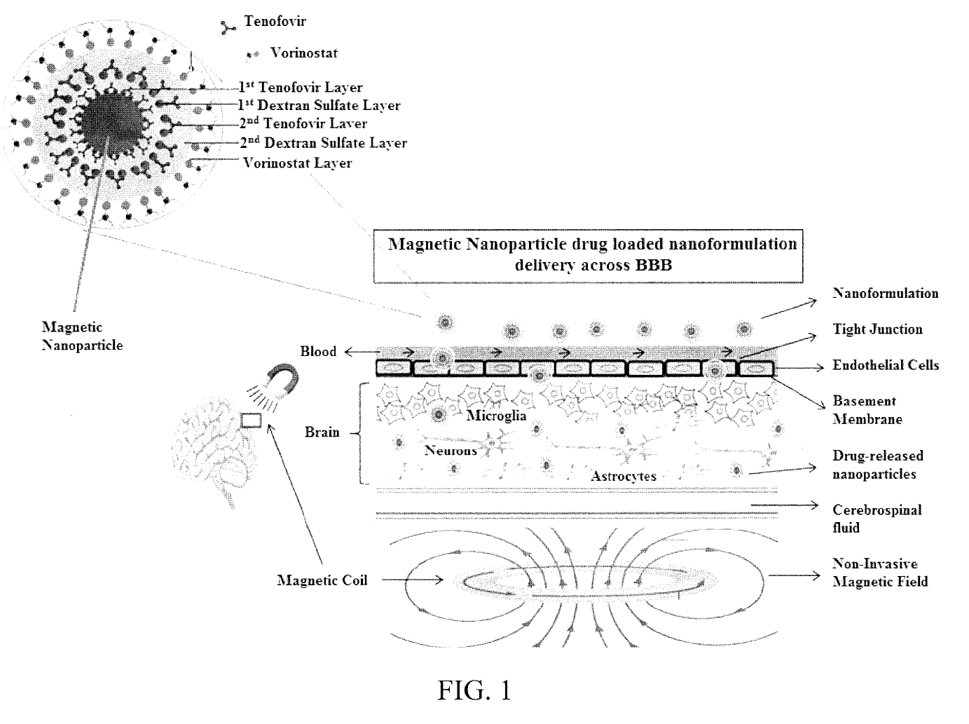

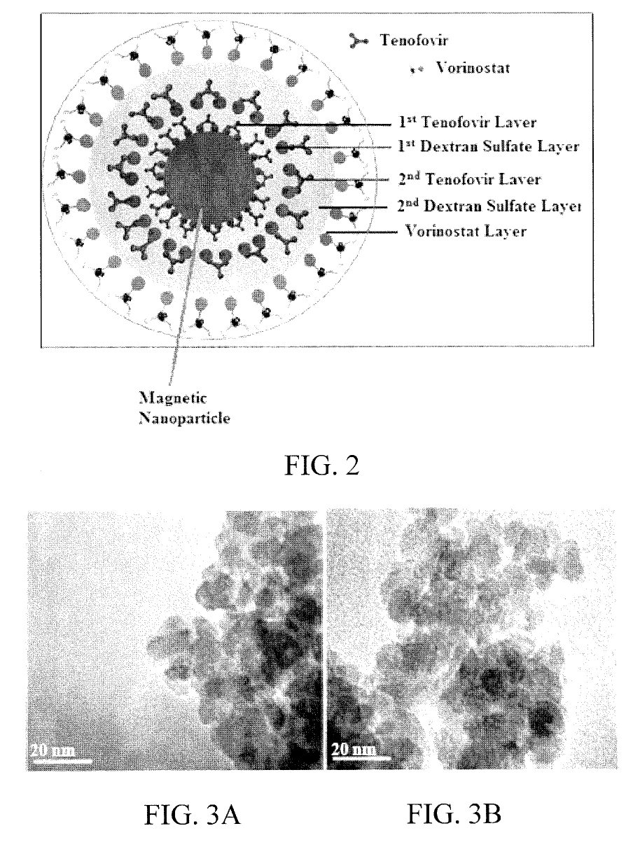

Advantageously, for the treatment of HIV-AIDS, specific embodiments of the nanoparticle drug delivery system provided herein comprise HIV latency activators packaged with MNPs in conjunction with an antiretroviral drug. Further, the combination drug system can be delivered across a BBB model and subsequently released in a sustained manner without compromising the integrity of the BBB and cell viability (see, for example, FIGS. 1 and 2).

### Active Agents

The terms “drug” and “active agent,” as used in the present application, include any natural or synthetic substance that has a physiological effect when administered to an animal. As used herein, the terms “drug” and “active agent” include therapeutic and diagnostic agents. The drug can be suitably employed in accordance with the invention with animals (subjects), particularly mammals including humans, veterinarian animals and farm animals. Drugs used in accordance with the subject invention can include those affecting, acting on, or being visualized at a desired target within, or on, the animal body, such as, for example, within the nervous system, including tumor tissue located therein.

In preferred embodiments of the invention, the drug to be delivered to the subject comprises one or more therapeutic agents and/or diagnostic agents. The nanoparticle drug delivery system can comprise one drug or more than one drug. If there are multiple drugs, preferably the drugs are compatible with each other in the same nanoparticle and/or nanoparticle drug delivery system and exhibit physiological effects that are not incompatible with each other. In certain embodiments, if more than one drug is present, the drugs exert a synergistic effect.

Specific examples of physiologically effective active agents and drugs, which do not restrict the present invention, are therapeutic agents selected from the group consisting of: drugs acting at synaptic sites and neuroeffector junctional sites; general and local analgesics; hypnotics and sedatives; drugs for the treatment of psychiatric disorders such as depression and schizophrenia; anti-epileptics and anticonvulsants; drugs for the treatment of Parkinson's and Huntington's disease, aging and Alzheimer's disease; excitatory amino acid antagonists, neurotrophic factors and neuroregenerative agents; trophic factors; drugs aimed at the treatment of CNS trauma or stroke; drugs for the treatment of addiction and drug abuse; drugs for the treatment of bacterial, viral and/or microbial infections, such as influenza viral infections, HIV, herpes, chicken pox, and the like; antacids and anti-inflammatory drugs; chemotherapeutic agents for parasitic infections and diseases caused by microbes; immunosuppressive agents and anti-cancer drugs; hormones and hormone antagonists; heavy metals and heavy metal antagonists; antagonists for non-metallic toxic agents; cytostatic agents for the treatment of cancer; diagnostic substances for use in nuclear medicine; immunoactive and immunoreactive agents; transmitters and their respective receptor agonists and receptor antagonists, their respective precursors and metabolites; transporter inhibitors; antibiotics; antispasmodics; antihistamines; antinauseants; relaxants; stimulants; sense and antisense oligonucleotides; cerebral dilators; psychotropics; antimanics; vascular dilators and constrictors; anti-hypertensives; drugs for migraine treatment; hypnotics, hyperglycemic and hypoglycemic agents; minerals and nutritional agents; anti-obesity drugs; anabolics; and anti-asthmatics.

### Encapsulating Bilayers

In one aspect, the present invention provides a nanoparticle drug delivery system comprising a magnetic nanoparticle (MNP) encapsulated by at least one bilayer coating comprising a layer of drug molecules and a layer of polymer. In some embodiments, the polymer may comprise polyelectrolytes.

In some embodiments, there can be more than one bilayer, with each bilayer additional to the first comprising a layer of drug molecules and a layer of a polymer, where the drug molecules can be the same as, or distinct from, the first drug molecules and the polymer can be the same as, or distinct from, the first polymer.

In certain embodiments, the drug and the polymer of the bilayer coating are oppositely charged. In specific embodiments, the polymer is negatively charged (polyanion). In other embodiments, the polymer is positively charged (polycation). The bilayer structure may be assembled such that the oppositely charged drug molecules and polymer adsorb onto the MNP in an alternating, layer-by-layer (LbL) self assembled manner.

Preferably, the polymer comprises biodegradable materials and may be selected from, for example, alginate, heparin, chondroitin sulfate A & B, collagen, gelatin-A, dextran sulfate, chitosan, hyaluronic acid, poly-L-lysine, protamine sulfate, poly-L-arginine, carboxymethyl cellulose, polyglutamic acid, albumin, dextran amine, DNA, RNA, and combinations thereof.

### Magnetic Nanoparticles

In certain embodiments, the MNP comprise iron oxide. In an exemplary embodiment the MNP comprise Fe₃O₄ and/or Fe₂O₃. Magnetite (Fe₃O₄) is a commonly used MNP in the field of biomedicine, mainly because of its biocompatibility.

The MNPs can be prepared using, for example, a coprecipitation method (such as that used in Sun, Y. et al., Colloids Surf A Physicochem Eng Asp. 2004; 245(1-3):15-19). Advantageously, this method is cost-effective for preparing nanoscopic magnetic particles. Synthesized MNPs may be subsequently tested for their drug-loading, in vitro release pharmacokinetics, cytotoxicity, and in vitro antiviral replication efficacy in central nervous system cells including, but not limited to, the primary human astrocytes (HAs) found in the brain. As an example, FIG. 3A shows a transmission electron microspy (TEM) image of Fe₃O₄ MNPs with sizes in the range of 10±3 nm.

### Layer-by-Layer Preparation

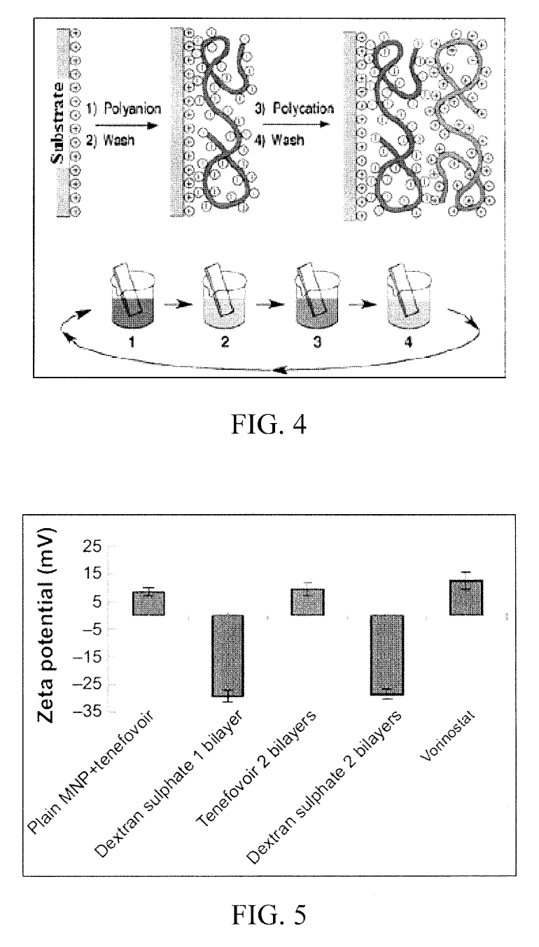

LbL assembly involves the spontaneous adsorption of alternating layers of positively and negatively charged species, allowing the design of functional surfaces and surface-based nanostructure in a “build-to-order” fashion (see, for example, FIG. 4). Advantageously, this technique is simple, fast, and versatile as a result of the electrostatic interaction between the oppositely charged layers. The rate of release can also be adjusted through shell thickness, which is a function of the number of adsorbed molecular layers. In practice, multi-layer build-up can be confirmed by monitoring the zeta-potential values, which reverse in polarity when the surface of the MNPs is sequentially coated with oppositely charged moieties.

In a further embodiment, the MNP-LbL assembly can be further encapsulated in a liposome to facilitate interaction between the drug delivery system and the blood, as well as protecting the drug against enzymatic activities in vivo for improved cargo delivery efficacy to a desired treatment area. Further, a drug delivery system modified with liposome can also help the incorporation of non-water-soluble drug molecules into the nanoparticles.

### Pharmaceutical Compositions

The nanoparticle drug delivery system of the invention can be delivered as part of a composition that further comprises a physiologically acceptable carrier and/or diluent allowing the transport of said nanoparticles to the target after administration.

The carrier and/or diluent can be any medium by which the desired purpose is achieved and which does not affect the capability of the nanoparticles to be directed to the desired target and to transport the drug(s) to this target for the desired pharmacological effect. Particularly, the carrier and/or diluent should not deteriorate the pharmacological potency of the drug and the capability of the nanoparticle delivery system to be directed to a desired target within or on the mammalian body.

Preferably, the carrier and/or diluent is selected from water, physiologically acceptable aqueous solutions containing salts and/or buffers and any other solution acceptable for administration to a subject. Such carriers and diluents are well known to a person skilled in this field and include, for example, distilled water, de-ionized water, pure or ultrapure water, saline, phosphate-buffered saline (PBS), and solutions containing usual buffers which are compatible with the other components of the drug targeting system provided herein.

### Conditions to be Treated

In one embodiment, a nanoparticle drug delivery system as described herein can be used to treat brain diseases selected from degenerative disorders, sensory and locomotor abnormalities, seizures, viral infections, immunological infections, mental and behavioral disorders, and localized central nervous system disease.

### Treatment of Viral Infections, Including HIV-AIDS

In a specific embodiment, the subject drug delivery system can be used to treat viral infections, including HIV-AIDS. Thus, in specific embodiments, the first drug in the at least one bilayer coating is an antiretroviral drug. A second bilayer can comprise a latency-activating drug. The antiretroviral drug may be selected from, for example, nucleoside reverse transcriptase inhibitors (NRTIs), non-nucleoside reverse transcriptase inhibitors (NNRTIs), nucleotide analog reverse transcriptase inhibitors (NtARTIs or NtRTIs), protease inhibitors (PIs), and integrase strand transfer inhibitors (INSTIs). The viral latency-activating drug may be selected from, for example, protein kinase C (PKC) agonists, histone deacetylase (HDAC) inhibitors, and nuclear factor kappa-light-chain-enhancer of activated B cells (NF-κB) etc.

In an exemplary embodiment, the drug in one bilayer coating is tenofovir (teno), an NRTI, and the drug in a second bilayer is vorinostat (vor), an HDAC inhibitor.

Advantageously, technologies provided herein may also be applied to other combinations of antiretroviral drugs including, but not limited to, nucleotide reverse transcriptase inhibitor+nucleotide reverse transcriptase inhibitor+protease inhibitor, and nucleotide reverse transcriptase inhibitor+protease inhibitor+integrase inhibitor. Further, in other embodiments, the nanoparticle drug delivery system may also be used to treat other types of viral infections using specific drug or drug combinations.

Latency-breaking and antiviral efficacy of the drug delivery system provided for treating HIV-AIDS can be determined via, for example, quantification of p24 antigen in a primary human astrocytes (HA) infection model, similar to what was described by Nair et al. (Nair M, Guduru R, Liang P, Hong J, Sagar V, Khizroev S. Externally controlled on-demand release of anti-HIV drug using magneto-electric nanoparticles as carriers. Nat Commun. 2013; 4:1707).

An exemplary embodiment of the present invention provides a formulation of the drug delivery system comprising an MNP core encapsulated by two bilayers, each comprising a layer of tenofovir and a layer of dextran sulfate (DS), wherein the MNP+tenofovir is positively charged and the DS is negatively charged. In a further embodiment, the bilayer-coated MNP is encapsulated by a layer of vorinostat, which is also positively charged. The zeta potential measurements of the MNPs following each adsorbed polyeletrolytic layer and the adsorbed layer of vorinostat are presented in FIG. 5.

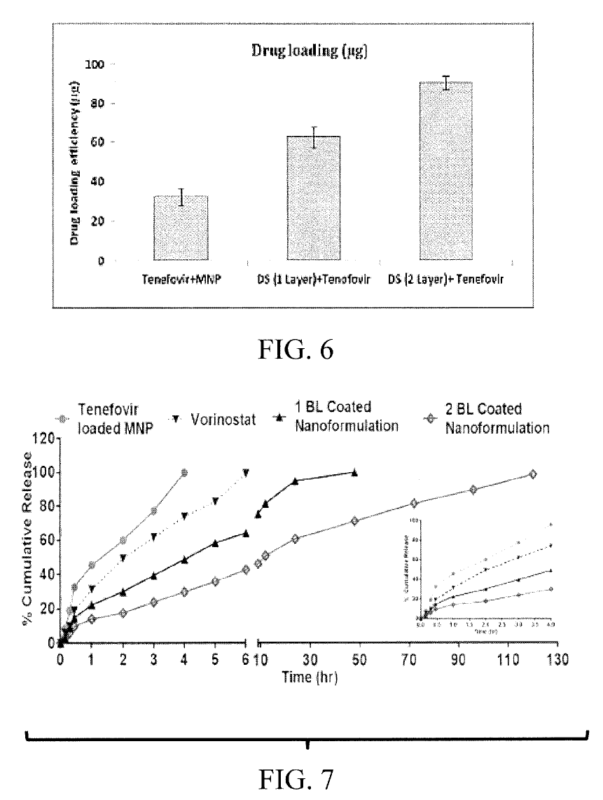

The drug release profile of an exemplary formulation, e.g., the {MNPs+(teno+DS)₂+vor} formulation, can be determined via an in vitro pharmacokinetic study in PBS (pH=7.4). As with the measurements of zeta potential, release profile of uncoated, 1 BL-coated, and 2BL-coated drug particles, respectively, can be compared to demonstrate improved drug release profile. Advantageously, LbL arrangements of drugs on MNPs can also increase the amount of drug loaded (see, for example, FIG. 6), providing sustained release over a longer period of time and potentially improving patient adherence to medication for antiretroviral HIV treatments, as well as treatments for HIV-associated neurocognitive disorder that results in substance abuse.

In certain embodiments, the concentration of the nanoparticle drug delivery system can be sustained for a longer period of time than nanoparticles comprising only one drug without the bilayer coating. In an exemplary embodiment, the period of sustained drug release in vitro for nanoparticles loaded with two bilayers of HIV-targeting drugs is about 5 days or more (see, for example, FIG. 7). In other embodiments, the period for sustained release is about 1 day, 3 days, 10 days, 2 weeks, a month, a year, or more.

### Localization to Target Area

In another aspect, the present invention provides a method of using the nanoparticle drug delivery system comprising administering the nanoparticle drug delivery system systemically and localizing the nanoparticles to the target treatment area.

In one embodiment, the localization of nanoparticles to the target treatment area may be accomplished by applying an external, non-invasive magnetic force. In another embodiment, the magnetic force may be remotely controlled (FIG. 1) for precise targeting of the drug to a desired treatment area.

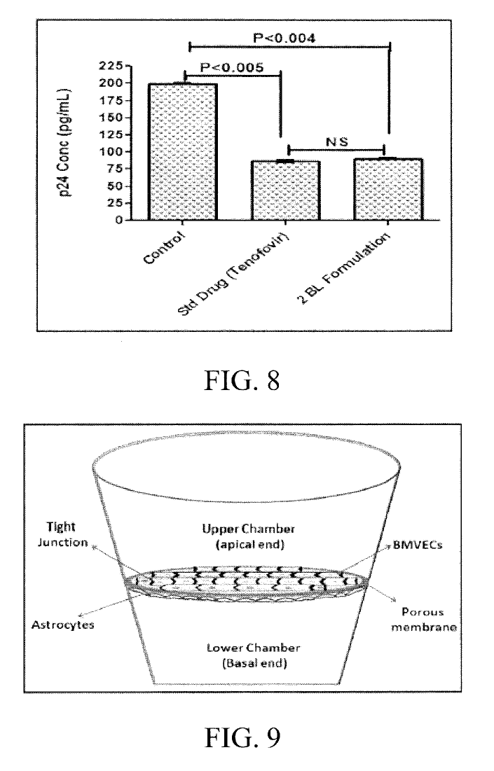

The integrity of the drug delivery system following the magnetically triggered release process can be evaluated using mass spectroscopy and analyzed for its antiviral efficacy using a p24 viral replication assay. FIG. 8 includes exemplary data demonstrating statistically equivalent p24 efficacy of standard tenofovir release (i.e., without magnetic trigger) and {MNPs+(teno+DS)₂+vor} release, respectively, after seven days post-infection, suggesting that the therapeutic integrity of the drug is not compromised by the magnetic force.

In one embodiment the invention provides a method of delivering pharmacologically active substances across the BBB. Thus, the invention can be used to treat a viral infection in the brain.

The blood-brain barrier, a highly selective permeability barrier separating blood from brain extracellular fluid, comprises, inter alia, endothelial cells connected by tight junctions and HAs. In certain embodiments, the bilayer formulations provided herein result in nanoparticles with sizes in 10 s of nanometers (FIGS. 3A and 3B), an improvement over past technologies that provided formulations encapsulated in liposomes with sizes on the order of 100 nm (Ding, H. et al., Nanotechnology. 2014; 25(5):055101; Kargol, A. et al., Advanced Magnetic Materials. Rijeka, Croatia: Intech; 2006:89-118; Caraglia, M. et al., Curr Cancer Drug Targets. 2012; 12(3):186-196; De Rosa, G. et al., Curr Drug Metab. 2012; 13(1):61-69; Kaushik, A. et al., Expert Opin Drug Deliv. 2014; 11(10):1635-1646). This improvement is of particular importance for drug delivery systems targeting infected tissues with tight junctions including, but not limited to, the BBB.

The term “tight junctions,” as used herein, refers to multiprotein complexes formed between tightly joined cells that selectively regulate the diffusion of ions and water-soluble molecules through the paracellular pathway. Tight junctions are characterized by their high electrical resistance, approximately on the order of 1000 ohms·cm² for structures such as the BBB. The nanoparticle drug delivery system of the subject invention can also be used to treat other tissues comprising cellular tight junctions.

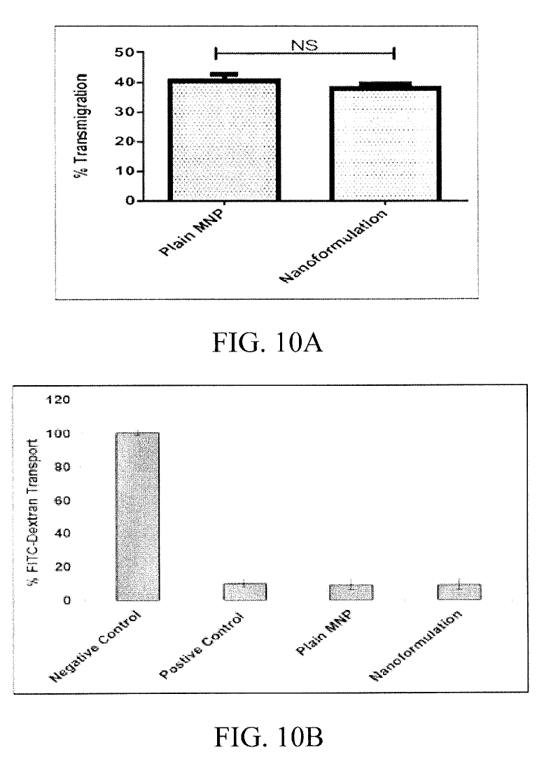

The transmigrability of the subject nanoparticle formulations can be evaluated using an in vitro BBB model comprising co-cultured primary human brain microvascular endothelial cells and HAs (FIG. 9) (Persidsky, Y. et al., J Immunol. 1997; 158(7):3499-3510). First, the integrity of the BBB prior to and following the treatment can be measured as a function of the transendothelial electrical resistance, a parameter measuring the intactness of the tight junctions between the endothelial cells. Similar before- and after-treatment values suggest the preservation of the permeability function of the BBB. Second, the efficacy of the formulation with respect to the transmigrability can be verified by measuring the iron concentration across the BBB after the application of external magnetic field exemplified by data shown in FIG. 10A.

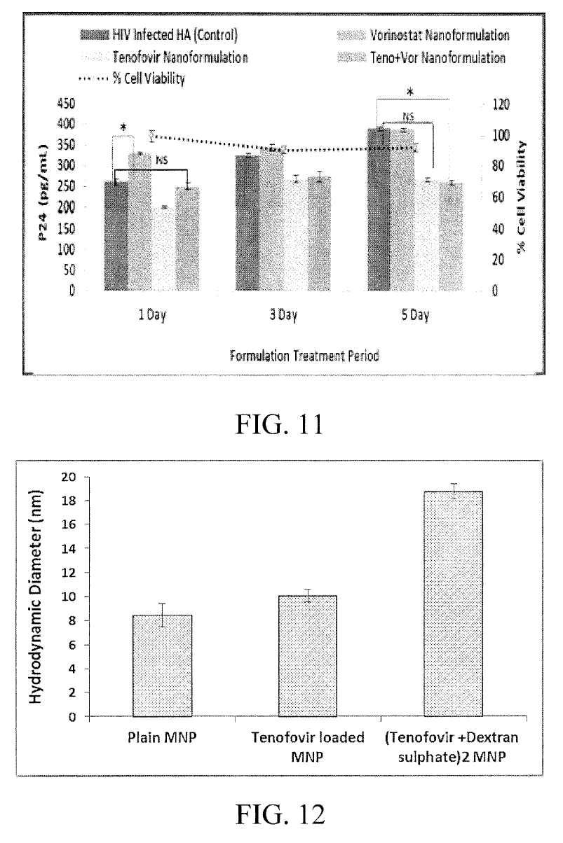

In a specific embodiment, the subject drug delivery system can be used to treat a person who has been diagnosed with HIV-AIDS. The combination of two distinct drugs, one being antiretroviral and another being latency-activating, provides the combined therapeutic effects of activating latent HIV expression and killing the virus. The therapeutic efficacy of the nanoparticle drug delivery systems comprising tenofovir or vorinostat, or both, may be evaluated using a standard p24 antigen level assessment of an HIV-infected HA (7-day infection) model over 5 days of treatment as shown in FIG. 11.

### Routes of Administration

The administration of the nanoparticle drug delivery system of the subject invention can be carried out generally in any desired manner or on any desired route of administration in order to achieve entry into the subject and transportation thereby to the targeted area (such as the BBB). Administration can be, for example, via oral, intravenous, subcutaneous, intramuscular, intranasal, pulmonal or rectal route. Systemic routes can be used in view of the ability to transport the drug targeting system to the site of treatment within or on the body via magnetic force.

### EXAMPLES

Following are examples that illustrate procedures for practicing the invention. These examples should not be construed as limiting.

Experiments were performed at least three times in duplicate unless otherwise indicated in the figure legends. The values obtained were averaged, and data are represented as the mean±standard error. All the data were analyzed using GraphPad Prism software. Comparisons between groups were performed using one-way analysis of variance. Differences were considered significant at p≦0.05.

### Example 1

### Synthesis of MNPs

The Fe₃O₄ MNPs were synthesized according to the co-precipitation method proposed by Sun et al. (Colloids Surf A Physicochem Eng Asp. 2004; 245(1-3):15-19) with minor modifications.

Briefly, a solution comprising approximately 3 mL FeCl₃ (about 0.487 g FeCl₃ dissolved in approximately 2 mol/L HCl) and about 10.33 mL H₂O was stir-mixed with about 2 mL Na₂SO₃ (about 0.126 g in about 2 mL water) drop by drop over the course of a minute. On the change in color from yellow to red-light yellow, this solution was mixed into approximately 80 mL NH₃+H₂O (about 0.85 mol/L) under vigorous stirring. A black precipitant quickly formed, which was allowed to crystallize further, for approximately 30 minutes, under continuous stirring. The suspension was washed, and formation of stable MNPs was achieved by adjusting the pH from about 3.0 to about 7.5 and the temperature from approximately 90° C. for the first 5 minutes to about 100° C. for about 1 hour.

A change in color from black to reddish-brown suggests the formation of compact MNPs, which are washed with water at least three times by the decantation method.

### Example 2

### Characterization of MNPs

The MNPs were characterized for size, shape, crystallinity, and drug loading capability.

Structural conformation of MNPs was determined by Shimadzu XRD-7000 diffractometer (Shimazdu, Tokyo, Japan). Transmission electron microscopy (TEM) of MNPs was performed with a Phillips CM-200 200 kV transmission electron microscope operated at 80 kV. In brief, one drop of MNPs was spread on carbon support film on 400 mesh Cu grids (type B; Ted Pella, Inc., Redding, Calif., USA). For better contrast during TEM imaging, samples on the grid were negatively stained with phosphotungstic acid (about 2.0% w/v with a pH of about 6.4) and dried at room temperature. The size distribution and surface charge measurement of MNPs were carried out at approximately 25° C. in dynamic laser scattering (Zetasizer NanoZS, Malvern). The measurement of super paramagnetism was carried out by a classical vibrating sample magnetometer (Model 4 HF VSM, ADE, Lowell, Mass., USA). The magnetic hysteresis loops of the Fe₃O₄ particles were measured between +1,200 and −1,200 Oersted at room temperature.

TEM images show that nanoparticles were in the range of 10±3 nm, as shown in FIGS. 3A and 3B, with excellent dispersion properties (polydispersion index=0.81) in aqueous medium.

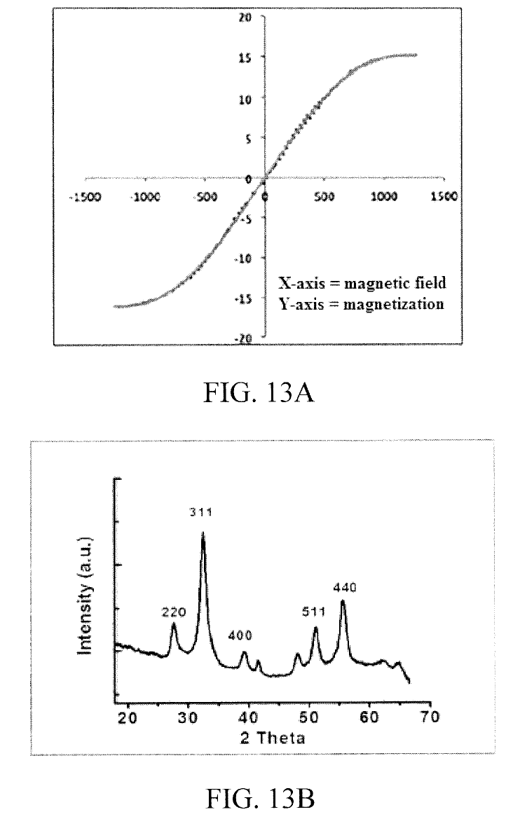

In addition, the effect of drug loading and LbL deposition on MNP with respect to its hydrodynamic diameter also was determined and shown in FIG. 12. Magnetic hysteresis loops for MNP displayed strong magnetic behavior (exhibited by a superparamagnetic property with no coercivity and remanence), when measured between +1,200 and −1,200 Oersted at room temperature (as shown in FIG. 13A). The crystal structure of synthesized magnetite particles was confirmed by X-ray diffraction (XRD) spectroscopic measurement (FIG. 13B). The X-ray spectrum consists of magnetite-specific peaks that correspond to the 220, 311, 400, 511, and 440 planes.

### Example 3

### Layer-by-Layer Coatings on MNPs

An LbL self-assembly technique was used for nanoparticle coatings (as shown in FIG. 4). The polyelectrolyte pairs used for this experiment were dextran sulphate (polyanion: approximately 2 mg/mL in about 0.15 M of NaCl; molecular weight: 500,000 g/mol) and tenofovir (polycation: approximately 1 mg/mL in PBS; molecular weight: 287.213 g/mol).

A solution of approximately 2 mg/mL concentration of dextran sulphate sodium (DS) was prepared with about 0.15 M of NaCl, with pH adjusted to 7.4. For the coating, approximately 100 μL DS was added to about 1 mg Teno-loaded MNPs and kept at room temperature for about 20 minutes with intermittent shaking. The nanoparticles were then centrifuged at 2,500 rpm for about 3 minutes to separate them from the unreacted DS solution and were washed with Milli-Q water. Later, approximately 100 μl (at about 1 mg/mL) positively charged drug (i.e., tenofovir) was added for second-layer deposition to nanocarriers and held for 20 minutes of intermittent shaking.

Finally, coated MNPs were washed twice with distilled water, using centrifugation.

A similar process can be repeated two times to have 2 bilayer coatings on MNPs. The surface charge of the MNPs can be measured, using the zeta potential analyzer, after rinsing and before the addition of each layer. Finally, a vorinostat (concentration=approximately 0.05 mg/mL) layer was deposited onto the bilayer [tenofovir+DS]₂ system. The final assembled nanoformulation was washed with distilled water, centrifuged, and subjected to release studies.

LbL assembly deposition was confirmed using zeta potential analysis. Results show that the zeta-potential value for MNPs coated with tenofovir was +8.5±2 mV, which reversed upon the adsorption of DS coating to −29.3±3 mV and kept on reversing on subsequent layer deposition, as shown in FIG. 5, thus confirming that layer deposition is taking place, as desired and predicted.

Drug loading results showed (FIG. 6) that application of 1 layer of DS leads to 1.9 times (from about 32 to about 62.5 μg/mg MNPs) higher drug encapsulation and, in the case of 2 bilayers (2BL), loading increases to 2.8 times (from about 32 to about 90.7 μg/mg MNPs).

### Example 4

### Activation of Latent HIV and Evaluation of In Vitro Antiviral Efficacy of Nanoformulation

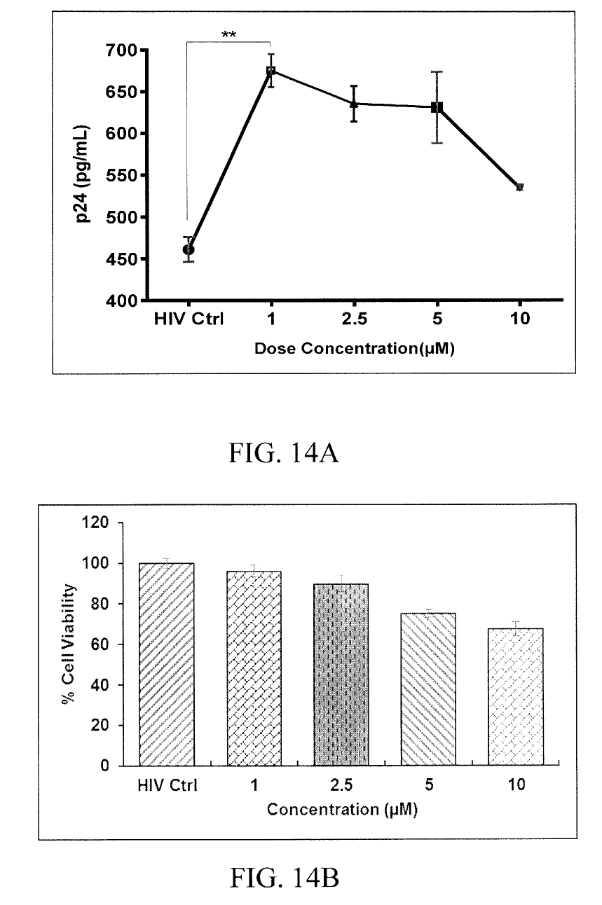

Latency-breaking and antiviral efficacy of the formulation was determined via quantification of p24 antigen in a primary human astrocytes (HA) infection model (Nair, M. et al., Nat Commun. 2013; 4:1707). Different concentrations (from about 1 to about 10 μM) of vorinostat (solution phase) were tested for the latency-breaking ability; the results showed that an approximately 1 μM concentration yields a significant difference in p24 value and was used for the final formulation development (FIG. 14A) and optimized dose in nontoxic to HA cells (FIG. 14B). The results show a good latency-breaking ability for vorinostat-loaded MNPs (about 20.5% increase in p24 levels) when compared with the positive control (HIV-infected HA cells), as shown in FIG. 11 on day 1 of formulation treatment. The sustained-release ability of the finally assembled formulation ([MNP+tenofovir]₂+vorinostat) was also evaluated over a treatment period of 5 days in the HA model.

To confirm the activation of latent cells and check the antiviral efficacy of the nanoformulation after the magnetic-triggered release process, a p24 antigen estimation was conducted. The cells were activated by treating them with polybrene (about 10 μg/mL) for approximately 6 hours before the infection. HAs (10×106 cells) were infected with HIV-1Ba-L (catalog #510; National Institutes of Health AIDS Research and Reference Reagent Program) overnight, as described by Atluri et al. (Atluri, V. S. R. et al., PLoS ONE. 2013; 8(4):e61399) washed with PBS and returned to culture with and without the drug-loaded formulation at equimolar concentrations. The culture supernatants were quantitated for p24 antigen, using an enzyme-linked immunosorbent assay kit (ZeptoMetrix, NY, USA) on the third, fifth, and seventh days post-infection, expressed as picograms per milliliter.

The results (FIG. 11) showed a good latency-breaking ability for vorinostat-loaded MNPs (about a 20.5% increase) when compared for p24 levels, with respect to positive control (i.e., the HIV-infected HAs). Furthermore, there was a significant suppression of viral load by about 33%, dropping from about 390 to about 260 pg/mL, of p24 level found after 7 days of HIV infection with 5 days of formulation treatment, which clearly shows that drug is released in a sustained manner over a period of 5 days and is able to activate latent cells and suppress viral replication.

### Example 5

### Time Kinetics and Drug-Binding Isotherm on MNPs

For determining the time kinetics of direct binding of tenofovir on MNPs, about 1 mg/mL tenofovir solution to about 1 mg MNPs was incubated in PBS buffer (pH=approximately 7.4) for 0.5, 1, 1.5, and 2 hours at room temperature. The amount of bound drug or percentage binding to the MNPs was determined by estimating the concentration of tenofovir in the unbound fraction (supernatant) of the mixture by the spectrophotometric method at an ultraviolet absorbance of 260 nm. In addition, the effect of different drug concentrations (from about 1 to about 4 mg/mL) with respect to drug binding was calculated. Similarly for vorinostat, time kinetics and percentage drug binding were calculated using ultraviolet spectrophotometry at an absorbance of 240 nm.

The pharmacokinetic release studies show that uncoated MNPs release 100% drug in 4 and 7 hours, respectively, for tenofovir and vorinostat. The application of LbL assembly (1 layer and 2BL) increases the tenofovir release duration from 48 to 120 hours, as shown in FIG. 7, and its inset, which show the initial 4 hour burst release data for all drug delivery system formulations.

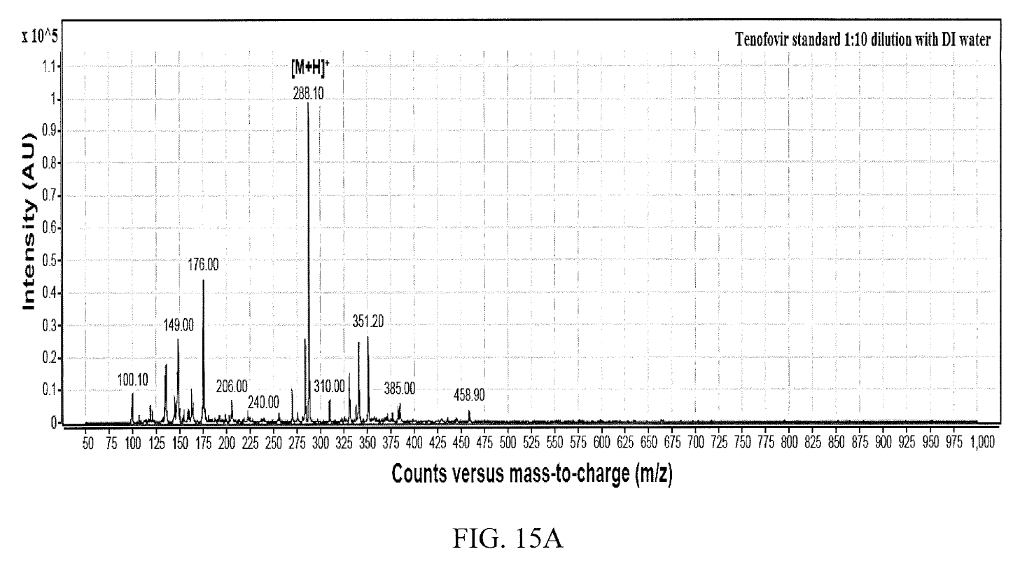

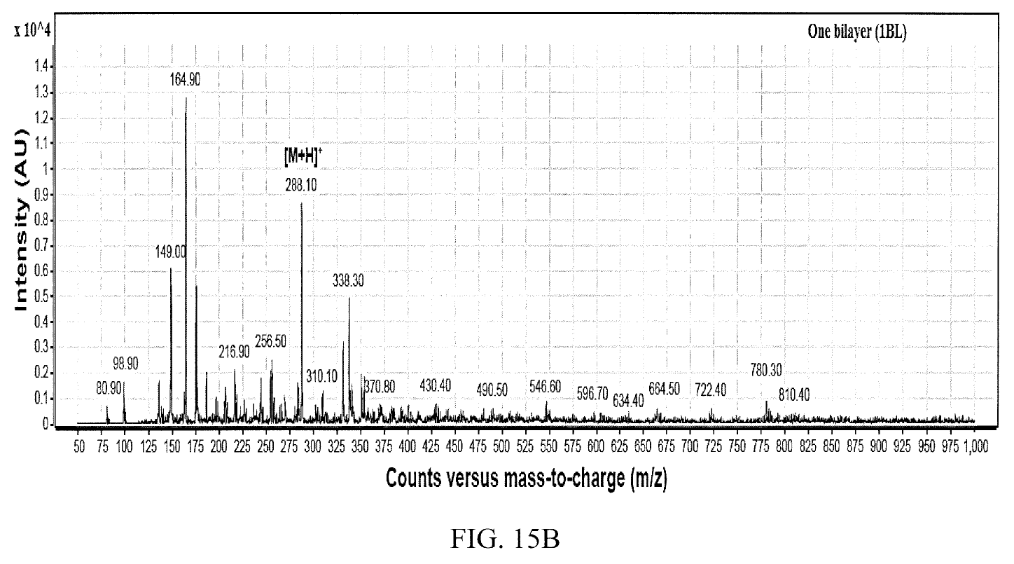

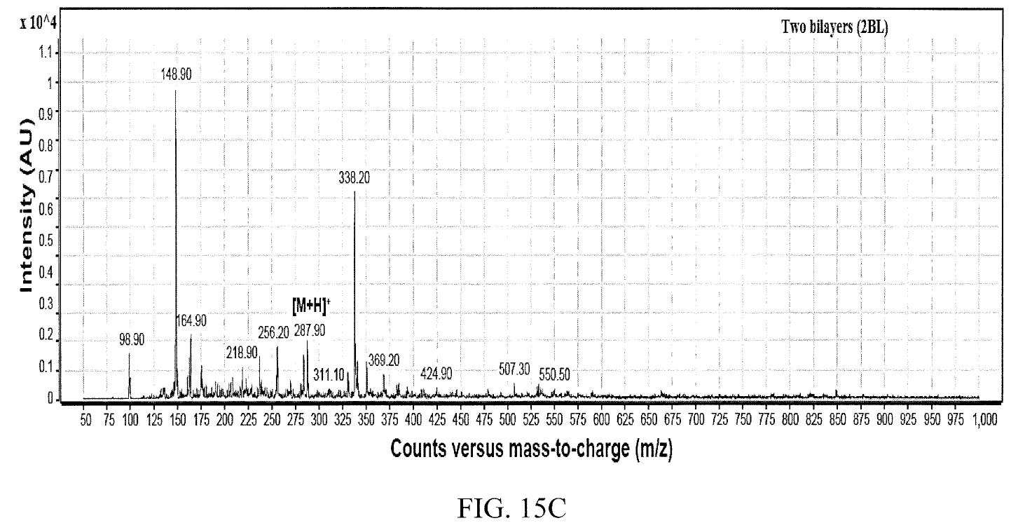

To confirm the structural and functional integrity of the drug after the release process, mass spectrometry analysis (details given in Table 1 below) of the drug (tenofovir) was performed before (standard drug) and after the release (results shown in FIGS. 15A-15C).

TABLE 1
The mass spectrometry instrumental parameters used for the
LC/MSD analyses (the General Screen Method) (Nair M, Guduru
R, Liang P, Hong J, Sagar V, Khizroev S. Externally controlled
on-demand release of anti-HIV drug using magneto-electric nanoparticles
as carriers. Nat Commun. 2013; 4: 1707.)

| | Value | |
|---|---|---|
| Source Parameter | | |
| Drying Gas Temp | 325° | C. |
| Gas Flow | 10 | psi |
| Nebulizer | 30 | psi |
| Sheath gas heater | 375° | C. |
| Sheath Gas Flow (l/min) | 11 | |
| Nozzle Voltage | 0 | V |
| Capillary Voltage (Vcap) | 3500 | V |
| Scan Parameters | | |
| Mass Range | 50-1000 | |
| Scan time | 350 | ms |
| Fragmentor voltage | 135 | V |
| Cell Acelerator voltage | 4 | V |

| | |
|---|---|
| Polarity | Negative |

### Example 6

### In Vitro BBB Preparation and Transmigration Assay

Primary human brain microvascular endothelial cells and HA cells were cultivated (ScienCell Research Laboratories, CA, USA). The BBB model was established as described by Persidsky et al. (J Immunol. 1997; 158(7):3499-3510). In brief, the in vitro BBB model was developed in a bicompartmental transwell culture plate (product 3415; Corning Life Sciences, Mexico). The upper chamber of this plate is separated from the lower one by a 10 μm thick polycarbonate membrane possessing 3.0-μm pores (FIG. 9). In a sterile 24-well cell culture plate with a pore density of about 2×106 pores/cm² and a cell growth area of approximately 0.33 cm², 2×105 human brain microvascular endothelial cells and HAs were grown to confluence on the upper chamber and underside of the lower chamber, respectively.

To assess the effect of MNP nanoformulation on the integrity of the in vitro BBB model, after transmigration assay, paracellular transport of fluorescein isothiocyanate (FITC)-dextran was measured (Saiyed Z M et al., Int J Nanomedicine. 2010; 5:157-166). Briefly, about 100 mg/mL of FITC-dextran (Sigma-Aldrich, St Louis, Mo., USA) was added to the upper chamber of the inserts and further incubated for 6 hours. Samples were collected from the bottom chamber after 6 hours, and relative fluorescence was measured at an excitation wavelength of about 485 nm and an emission wavelength of 520 nm, using a Synergy HT multimode microplate reader (BioTek Instru-ments, Inc., Winooski, Vt., USA) multimode microplate reader instrument. FITC-dextran transport was expressed as percentage FITC-dextran transported across the BBB into the lower compartment compared with negative control. Intactness of in vitro BBB was determined by measuring the transendothelial electrical resistance (TEER), using Millicell ERS microelectrodes (Millipore).

Transmigration study of drug-loaded MNP was conducted on the fifth or sixth day of the BBB culture, when ideal integrity of this membrane was achieved, as established by TEER measurement. MNP formulation was added to the apical chamber and incubated at approximately 37° C. in the presence or absence of a magnetic force of about 0.08 T placed externally, below the transwell basolateral chamber. MNP drug delivery system was collected from lower chambers at 6 hours, and percentage transmigration was analyzed at different points, using an ammonium thiocyanate-based photometric assay (Ding, H. et al., Nanotechnology. 2014; 25(5):055101).

As shown in Table 2, initial TEER values of all treatment groups were similar to 200+5 ohms·cm², which is the standard value of an in-vitro monolayer BBB model. Following the application of external magnetic force, very little differences were found in the TEER values when compared with the control with the uncoated MNPs, the unloaded MNPs, and the coated drug-loaded MNPs.

TABLE 2
Intactness of BBB tight junctions before
and after magnetic treatment.

| | TEER values (ohms · cm²) | | | |
|---|---|---|---|---|
| | Untreated | | | |
| | Well | Plain | Drug Loaded | 2BL MNP |
| | (Control) | MNP | MNP | Formulation |
| Before | 198.2 ± 7.9 | 201.2 ± 6.5 | 197.2 ± 5.9 | 194.3 ± 5.6 |
| treatment | | | | |
| After | 192.6 ± 5.8 | 198.4 ± 7.5 | 190.2 ± 6.2 | 196.2 ± 4.3 |
| Magnetic | | | | |
| treatment | | | | |

Formulation ability with respect to BBB transmigration was verified by measuring the iron concentration after application of the external magnetic field. As evident from FIG. 10A, 40%±3% of plain MNP versus 31.8%±3.5% of final nanoformulation {MNP+(tenofovir+DS)₂+vorinostat} crosses the BBB in the presence of about 0.8 T magnetic fields in 6 hours.

Without application of external magnetic force, only 2%±1.5% of the formulations were able to cross the BBB in 6 hours. Furthermore, the integrity of BBB was evaluated using paracellular transport of FITC-dextran, as described earlier, where the FITC molecule was used as a detection moiety to confirm the membrane intactness. FIG. 10B shows that only 7-8%±2% of the FITC molecule was detected in the transmigration (apical to basolateral chamber) in the entire drug delivery system treatment sample compared with approximately 100% in the case of negative control (without any BBB cells), thus showing that transmigration of MNP drug delivery system under external magnetic field does not affect the BBB integrity.

### Example 7

### Intracellular Uptake Analysis

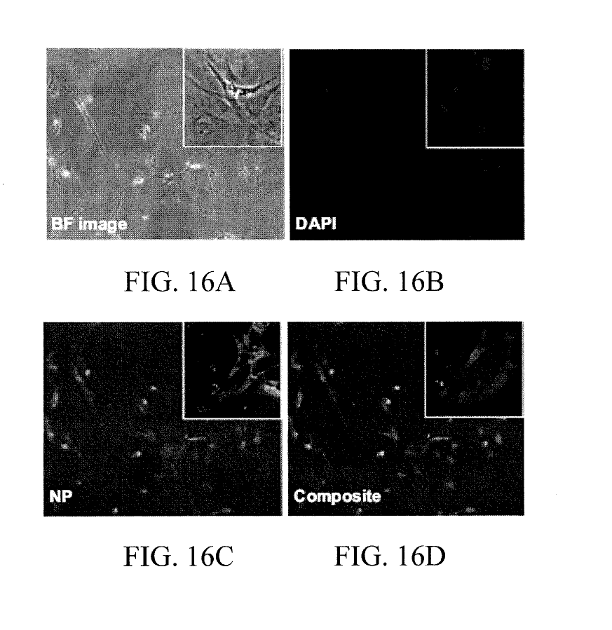

Qualitative and quantitative analysis of cellular uptake was performed using the same range of formulation concentrations (from about 50 to about 200 μg/mL). Confocal microscopy was used to visualize the MNP formulation particle uptake at different points (ranging from 6 to 24 hours), and confocal images were obtained at 488 nm with an argon-ion laser. FIGS. 16A-16D represent the cellular uptake of formulation in HAs after 6 hours of formulation treatment at approximately 100 μg/mL concentration.

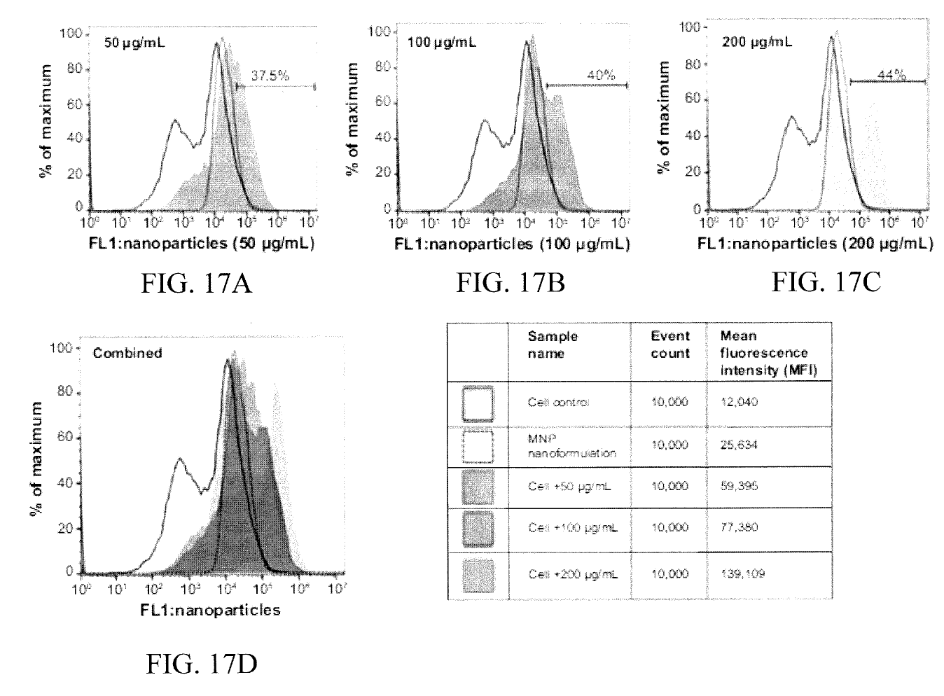

For quantitative analysis, a flow cytometer was used. HAs were treated with different concentrations of FITC-tagged formulation as specified above. The cells were then harvested at 24 hours after treatment and were counted. Equal amounts of cells (1×10⁶) were aliquoted in 12×75 mm polystyrene falcon tubes (catalog 352058; BD Biosciences, San Jose, Calif., USA) and fixed with Cytofix solution (BD Bioscience). Cells were acquired on an Accuri C6 flow cytometer (BD Accuri, Ann Arbor, Mich., USA) and analyzed with FlowJo software (Tree Star, Inc., Ashland, Oreg., USA). A total of 10,000 events were collected for each sample. Cells were gated on the basis of cells with and without formulation controls. Cells positive for formulation are shown in FIGS. 17A-17D as shaded histograms with shifted mean fluorescence intensity (MFI) compared with the controls.

The results show a concentration-dependent increase in the percentage of cells positive for FITC-tagged formulation: as the concentration increased from about 50 to about 200 μg/mL, there was a 1.25-fold (i.e., from about 35.7% to about 44%) increase in the percentage of cells positive for FITC-tagged formulation, as shown in FIGS. 17A-17D after 24 hours of treatment.

Furthermore, when analyzed with respect to the MFI, as shown in the legend, there was a significant concentration-dependent increase in MFI. For instance, a MFI of about 59,395 was observed for the approximately 50 μg/mL concentration, which increased to about 139,109 (i.e., 2.3-fold) when concentration was increased to about 200 μg/mL. Overall, the increase in MFI correlated with an increase in the percentage of cells positive for FITC-tagged formulation, and both increases were concentration-dependent.

### Example 8

### In-Vitro & In Vivo Cytotoxicity

In vitro cytotoxicity was assessed by MTT cell viability assay, using a CellTiter 96 Aqueous one-solution cell proliferation assay kit (catalog G3580; Promega, USA) (Nair, M. et al., Nat Commun. 2013; 4:1707). HAs were seeded in 96-well tissue plates at a density of 5×103 cells per well. After 24 hours of incubation at about 37° C., culture medium was replaced with about 100 μL fresh media containing different concentrations of Fe₃O₄ (from about 50 to about 250 μg/mL). Twenty microliters MTT solution (about 5 mg/mL in PBS) were added into each well after 24 hours post-treatment and incubated at approximately 37° C. for 2 hours. Finally, approximately 100 μL stop solution (about 20% sodium dodecyl sulfate in about 50% dimethyl formamide) was added into each well, and absorbance was recorded at 550 nm by a microplate reader (Synergy HT; BioTek Instruments, Inc.).

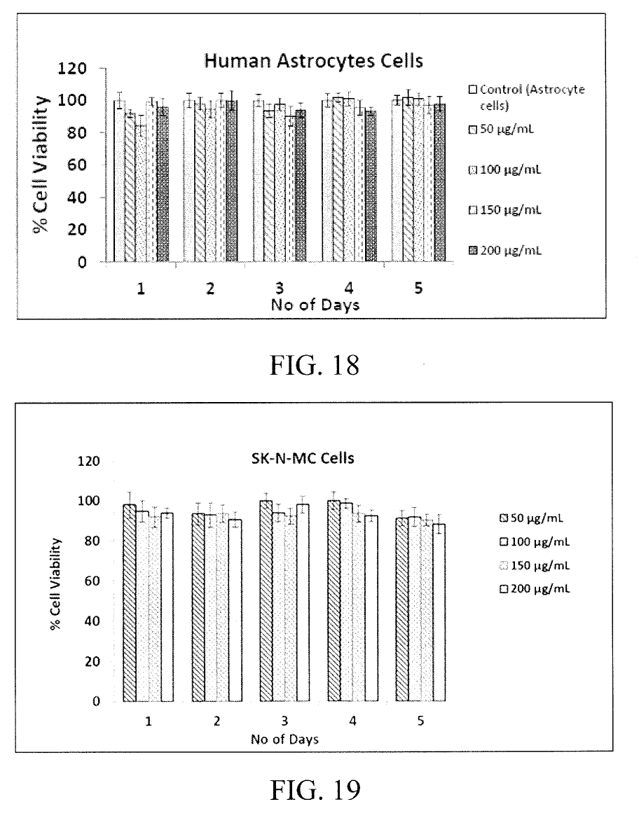

Results show (FIG. 18) that treatments with different concentrations (from about 50 to about 200 μg/mL) of the formulations do not show any cytotoxicity, and the percentage of viable cells was similar to untreated control, suggesting that doses of formulation are nontoxic and have no significant safety concerns. Experiments were also conducted in neuronal cells and peripheral cells, and there were no cytotoxic effects observed (data shown in FIG. 19 for neuronal cells).

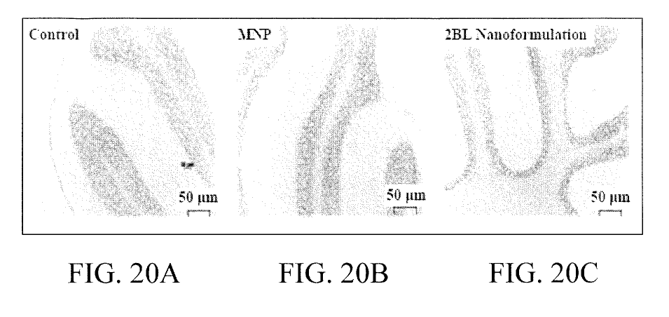

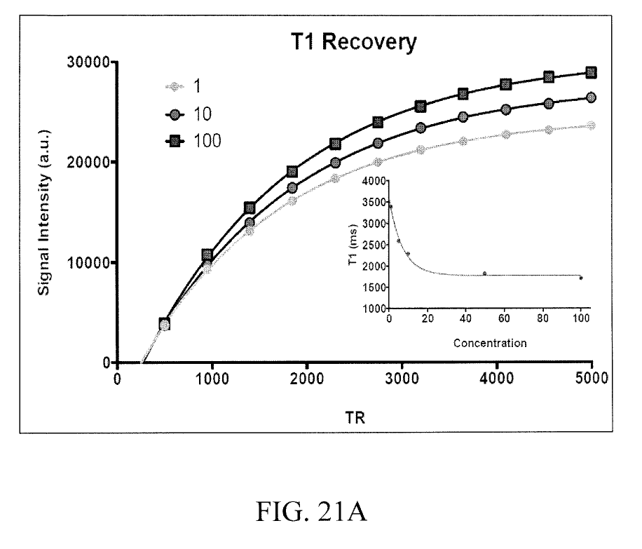

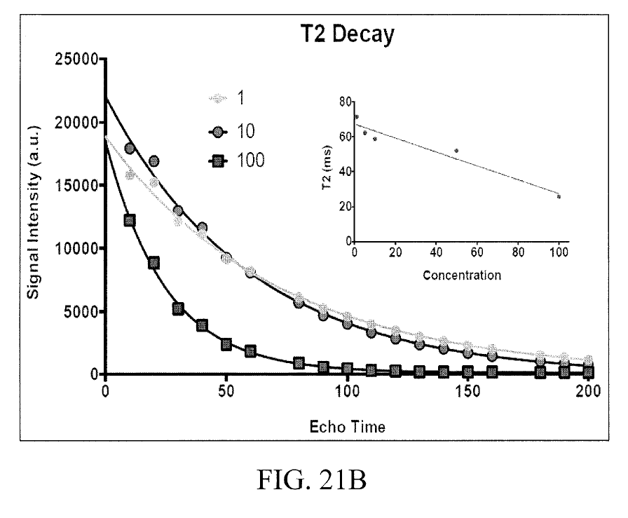

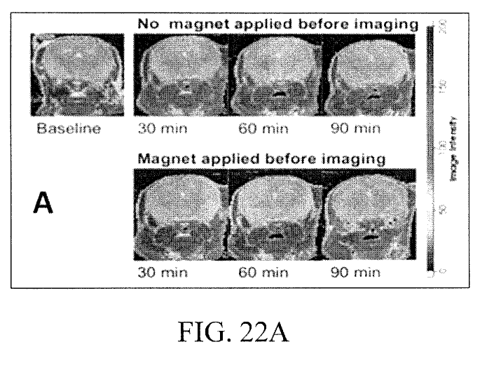

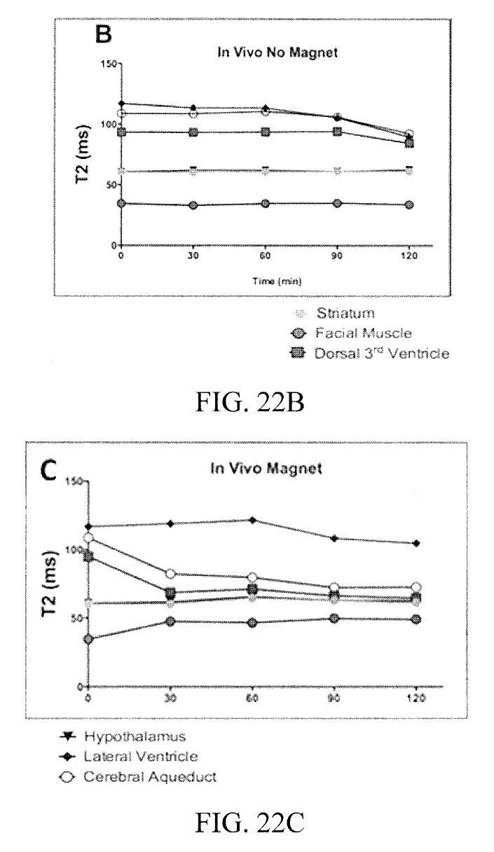

In vivo cytotoxicity of the nanoformulation was evaluated using dose of approximately 20 mg/kg with saline (about 0.9%) as control. H & E staining did not demonstrate any recruitment of macrophages or other immune cells in the brain of BALC-c mice (FIGS. 20A-20C). Results indicated that there was no cytotoxicity resulting from the drug delivery system in the mice model. Also, blood cytotoxicity of the drug delivery system was evaluated (Table 3), indicating that test level of the control and the drug were within the standard, prescribed range.

TABLE 3
An exemplary drug delivery system's in vivo blood cytotoxicity
analysis in BALB/C mice treated with approximately 20 mg/kg
of [MNP + (teno + DS)₂] nanoformulation,
The treatment period was approximately 48 hours.

| Renal/Hepatic | Test level | | | |
|---|---|---|---|---|
| Function | Control | Treated* | Ref. range | |
| ALBUMIN | 3.2 ± 0.15 | 3.1 ± 0.10 | 2.5-4.6 | g/dL |
| CARBON | 16.9 ± 0.45 | 16.5 ± 0.55 | 20.0-35.0 | mmol/L |
| DIOXIDE | | | | |
| CHLORIDE | 104.9 ± 3.8 | 102 ± 4.4 | 81.0-115 | mmol/L |
| CREATININE | 0.29 ± 0.09 | 0.35 ± 0.10 | 0.20-0.90 | mg/dL |
| JAFFE | | | | |
| GLUCOSE | 186 ± 12.48 | 196 ± 15.74 | 140-263 | g/dL |
| PHOSPHORUS | 10.8 ± 1.1 | 11.0 ± 1.4 | 5.5-12.9 | mg/dL |
| POTASSIUM | 8.79 ± 0.85 | 9 ± 0.94 | 4-10.5 | mmol/L |
| SODIUM | 152 ± 11.24 | 160.4 ± 14.26 | 110-195 | mmol/L |
| BUN | 21 ± 1.8 | 24 ± 3.5 | 9-33 | mg/dL |
| ALT | 33 ± 8.8 | 31 ± 7.4 | 17-77 | U/L |
| AST | 87 ± 18.5 | 90 ± 10.69 | 52-298 | U/L |
| BILIRUBIN | 0.1 ± 0.02 | 0.15 ± 0.01 | 0.0-0.9 | mg/dL |
| TOTAL | | | | |
| ALKALINE | 103 ± 1.8 | 119 ± 2.6 | 35-222 | U/L |
| PHOSPHATASE | | | | |
| PROTEIN | 5.1 ± 1.8 | 3.9 ± 2.4 | 3.9-6.4 | mg/dL |
| TOTAL | | | | |

The examples and embodiments described herein are for illustrative purposes only and various modifications or changes in light thereof will be suggested to persons skilled in the art and are included within the spirit and purview of this application. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

The description herein of any aspect or embodiment of the invention using terms such as “comprising”, “having”, “including” or “containing” with reference to an element or elements is intended to provide support for a similar aspect or embodiment of the invention that “consists of”, “consists essentially of”, or “substantially comprises” that particular element or elements, unless otherwise stated or clearly contradicted by context (e.g., a composition described herein as comprising a particular element should be understood as also describing a composition consisting of that element, unless otherwise stated or clearly contradicted by context).

The term “consisting essentially of,” as used herein, limits the scope of the ingredients and steps to the specified materials or steps and those that do not materially affect the basic and novel characteristic(s) of the present invention, i.e., compositions and methods for decellularization of tissue grafts. For instance, by using “consisting essentially of,” the compositions do not contain any unspecified ingredients including, but not limited to, surfactants that have a direct beneficial or adverse effect on decellularization of tissue.

All patents, patent applications, provisional applications, and publications referred to or cited herein (including those in the “References” section) are incorporated by reference in their entirety, including all figures and tables, to the extent they are not inconsistent with the explicit teachings of this specification.

### REFERENCES

1. Williams I, Churchill D, Anderson J, et al. British HIV Association guidelines for the treatment of HIV-1-positive adults with antiretroviral therapy 2012. HIV Med. 2012; 13(suppl 2):1-85.
2. Garcia de Olalla P, Knobel H, Carmona A, Guelar A, López-Colomés J L, Caylà J A. Impact of adherence and highly active antiretroviral therapy on survival in HIV-infected patients. J Acquir Immune Defic Syndr. 2002; 30(1):105-110.
3. Nowacek A, Gendelman H E. NanoART, neuroAIDS and CNS drug delivery. Nanomedicine (Lond). 2009; 4(5):557-574.
4. Bangsberg D R, Perry S, Charlebois E D, et al. Non-adherence to highly active antiretroviral therapy predicts progression to AIDS. AIDS. 2001; 15(9):1181-1183.
5. Adherence to HIV Antiretroviral Therapy. HIV InSite Knowledge Base Chapter; 2005 [reviewed January 2006]. Available from: http://hivinsite.ucsf.edu/insite?page=kb-03-02-09. Accessed Dec. 20, 2014.
6. Kelly J A, Kalichman S C. Behavioral research in HIV/AIDS primary and secondary prevention: recent advances and future directions. J Consult Clin Psychol. 2002; 70(3):626-639.
7. Martin L R, Williams S L, Haskard K B, Dimatteo M R. The challenge of patient adherence. Ther Clin Risk Manag. 2005; 1(3):189-199.
8. Kovochich M, Marsden M D, Zack J A. Activation of latent HIV using drug-loaded nanoparticles. PLoS ONE. 2011; 6(4):e18270.
9. Archin N M, Espeseth A, Parker D, Cheema M, Hazuda D, Margolis DM. Expression of latent HIV induced by the potent HDAC inhibitor suberoylanilide hydroxamic acid. AIDS Res Hum Retroviruses. 2009; 25(2):207-212.
10. Thomas SA. Anti-HIV drug distribution to the central nervous system. Curr Pharm Des. 2004; 10(12):1313-1324.
11. Ayre SG. New approaches to the delivery of drugs to the brain. Med Hypotheses. 1989; 29(4):283-291.
12. Bhaskar S, Tian F, Stoeger T, et al. Multifunctional Nanocarriers for diagnostics, drug delivery and targeted treatment across blood-brain barrier: perspectives on tracking and neuroimaging. Part Fibre Toxicol. 2010; 7(1):3.
13. Pardridge WM. Blood-brain barrier delivery. Drug Discov Today. 2007; 12(1-2):54-61.
14. Sagar V, Pilakka-Kanthikeel S, Pottathil R, Saxena S K, Nair M. Towards nanomedicines for neuroAIDS. Rev Med Virol. 2014; 24(2):103-124.
15. Saiyed Z M, Gandhi N H, Nair M P. Magnetic nanoformulation of azidothymidine 5′-triphosphate for targeted delivery across the blood-brain barrier. Int J Nanomedicine. 2010; 5:157-166.
16. Varatharajan L, Thomas SA. The transport of anti-HIV drugs across blood-CNS interfaces: summary of current knowledge and recommendations for further research. Antiviral Res. 2009; 82(2):A99-A109.
17. Boffito M, Jackson A, Owen A, Becker S. New approaches to antiretroviral drug delivery: challenges and opportunities associated with the use of long-acting injectable agents. Drugs. 2014; 74(1):7-13.
18. Ding H, Sagar V, Agudelo M, et al. Enhanced blood-brain barrier transmigration using a novel transferrin embedded fluorescent magneto-liposome nanoformulation. Nanotechnology. 2014; 25(5):055101.
19. Nair M, Guduru R, Liang P, Hong J, Sagar V, Khizroev S. Externally controlled on-demand release of anti-HIV drug using magneto-electric nanoparticles as carriers. Nat Commun. 2013; 4:1707.
20. Pilakka-Kanthikeel S, Atluri VSR, Sagar V, Saxena S K, Nair M. Targeted brain derived neurotropic factors (BDNF) delivery across the blood-brain barrier for neuro-protection using magnetic nano carriers: an in vitro study. PLoS ONE. 2013; 8(4):e62241.
21. Wohlfart S, Gelperina S, Kreuter J. Transport of drugs across the blood-brain barrier by nanoparticles. J Control Release. 2012; 161(2):264-273.
22. Sun Y, Ma M, Zhang Y, Gu N. Synthesis of nanometer-size maghemite particles from magnetite. Colloids Surf A Physicochem Eng Asp. 2004; 245(1-3):15-19.
23. Decher G. Fuzzy nanoassemblies: Toward layered polymeric multicomposites. Science. 1997; 277(5330):1232-1237.
24. Atluri V S R, Kanthikeel S P, Reddy P V, Yndart A, Nair M P. Human synaptic plasticity gene expression profile and dendritic spine density changes in HIV-infected human CNS cells: role in HIV-associated neurocognitive disorders (HAND). PLoS ONE. 2013; 8(4):e61399.
25. Toduka Y, Toyooka T, Ibuki Y. Flow cytometric evaluation of nanoparticles using side-scattered light and reactive oxygen species-mediated fluorescence-correlation with genotoxicity. Environ Sci Technol. 2012; 46(14):7629-7636.
26. Persidsky Y, Stins M, Way D, et al. A model for monocyte migration through the blood-brain barrier during HIV-1 encephalitis. J Immunol. 1997; 158(7):3499-3510.
27. Gandhi N, Saiyed Z M, Napuri J, et al. Interactive role of human immunodeficiency virus type 1 (HIV-1) Glade-specific Tat protein and cocaine in blood-brain barrier dysfunction: implications for HIV-1-associated neurocognitive disorder. J Neurovirol. 2010; 16(4):294-305.
28. Atluri V S, Pilakka-Kanthikeel S, Samikkannu T, et al. Vorinostat positively regulates synaptic plasticity genes expression and spine density in HIV infected neurons: role of nicotine in progression of HIV-associated neurocognitive disorder. Mol Brain. 2014; 7(1):37.
29. Jayant R. Layer-by-Layer (LbL) assembly of anti HIV drug for sustained release to brain using magnetic nanoparticles. J Neuroimmune Pharmacol. 2014; 9(1):25.
30. Jayant R D, McShane M J, Srivastava R. Polyelectrolyte-coated alginate microspheres as drug delivery carriers for dexamethasone release. Drug Deliv. 2009; 16(6):331-340.
31. Kargol A, Malkinski L, Caruntu G. Biomedical applications of multiferroic nanoparticles. In: Malkinski L, editor. Advanced Magnetic Materials. Rijeka, Croatia: Intech; 2006:89-118.
32. Caraglia M, De Rosa G, Salzano G, et al. Nanotech revolution for the anti-cancer drug delivery through blood-brain barrier. Curr Cancer Drug Targets. 2012; 12(3):186-196.
33. De Rosa G, Salzano G, Caraglia M, Abbruzzese A. Nanotechnologies: a strategy to overcome blood-brain barrier. Curr Drug Metab. 2012; 13(1):61-69.
34. Kaushik A, Jayant R D, Sagar V, Nair M. The potential of magneto-electric nanocarriers for drug delivery. Expert Opin Drug Deliv. 2014; 11(10):1635-1646.
35. Jayant R D, Srivastava R. Dexamethasone release from uniform sized nanoengineered alginate microspheres. J Biomed Nanotechnol. 2007; 3(3):245-253.
36. Kong S. D. et al., Magnetic targeting of nanoparticles across the intact blood-brain barrier. Journal of Controlled Release 164 (2012) 49-57.
37. Zainulabedin S et al. Magnetic nanoformulation of azidothymidinc 5′-triphosphate for targeted delivery across the blood-brain barrier. International Journal of Nanomedicine 5 (2010), 157-66.
38. Zainulabedin S et al., AZT 5-triphosphate nanoformulation suppresses human immunodeficiency virus type 1 replication in peripheral blood mononuclear cells. Journal of NeuroVirology 15 (2009), 343-347.
39. Christopher J. Destach. Brain as an HIV sequestered site: use of nanoparticles as a therapeutic option. Progress in Brain Research. 180 (2009) (Book Chapter ISSN: 0079-6123), DOI: 10.1016/50079-6123(08)80012-X.
40. Pathan et al. CNS Drug Delivery Systems: Novel Approaches. Recent Patents on Drug Delivery & Formulation, 3 (2009) 71-89.
41. Begley D. J, Delivery of therapeutic agents to the central nervous system: the problems and the possibilities. Pharmacology & Therapeutics 104 (2004) 29-45.
42. Chaowanachan T et al, Drug Synergy of Tenofovir and Nanoparticle-Based Antiretrovirals for HIV Prophylaxis. PLoS ONE 8 (2013): e61416. doi:10.1371/journal.pone.0061416.
43. Ojewole E et al, Exploring the use of novel drug delivery systems for antiretroviral drugs. European Journal of Pharmaceutics and Biopharmaceutics, 70 (2008) 697-710.
44. McGee B et al., HIV Pharmacology: Barriers to the Eradication of HIV from the CNS. HIV Clinical Trials 7 (2006) 142-153.
45. Shenoy D. B. Et al., Layer-by-Layer Engineering of Biocompatible, Decomposable Core-Shell Structures. Biomacromolecules 4 (2003) 265-272.
46. Cardoso F. L. et al, Looking at the blood-brain barrier: Molecular anatomy and possible investigation approaches. Brain Research reviews 64 (2010) 328-363.
47. Gerson T et al., Nano-NRTIs demonstrate low neurotoxicity and high antiviral activity against HIV infection in the brain. Nanomedicine: Nanotechnology, Biology, and Medicine (http://dx.doi.org/10.1016/j.nano.2013.06.012).
48. Yang H., Nanoparticle-mediated Brain-Specific Drug Delivery, Imaging, and Diagnosis. Pharmaceutical Research. 27 (2010) 1759-1771.
49. Rao K. S. et al., Targeting anti-HIV drugs to the CNS. Expert Opion Drug Delivery. 6 (2009), 771-784.
50. Walz A (8629114): Compositions and methods for transport across the blood brain barrier.
51. Khizroev S. & Nair M (US 20130317279 A1): Method for Non-Invasive Brain Stimulation.
52. Nair M & Saiyed Z. M (US 20110213193 A1): Magnetic Nanodelivery of Therapeutic Agents Across the Blood Brain Barrier.
53. Akhtari M. and Engel J (WO 2009123734 A1): Functionalized magnetic nanoparticles and methods of use thereof.
54. Cheon J. W. & Lee J. H (WO 2009136763 A2): Nanoparticles for penetration of blood-brainbarrier.
55. Krol S. & Lopez-Viota G. L (WO 2009141329 A1): Polyelectrolyte-encapsulated gold nanoparticles capable of crossing blood-brainbarrier.
56. Akhtari M (WO 2012145169 A2):Functionalized magnetic nanoparticles anduse in imaging amyloid deposits and neurofibrillary tangles.
57. Wu X. Y & Shalviri A (WO 2013127004 A1): Polymeric nanoparticles useful in theranostics.
58. Martel S. & Nasrollan S. (WO 2013170379 A1): Drug delivery across the blood-brain barrierusing magnetically heatable entities.
59. Huwyler J. & Pardridge W. M. (WO 1998022092 A1): Transport of liposomes across the blood-brain barrier.
60. Sung H. W. and Tu H. (U.S. Pat. No. 7,879,313 B1): Nanoparticles for protein drug delivery.
61. Gendelman H. E et al (WO 2013158549 A1): Small magnetite therapeutics and methods of use thereof.
62. Saiyed Z. M. et al., Int J Nanomedicine. 2010; 5:157-166.

What is claimed is:

1. A drug delivery system, comprising a magnetic nanoparticle (MNP) encapsulated by at least one bilayer coating, wherein the bilayer coating comprises a layer of first drug molecules and a layer of a first polymer.

2. The system, according to claim 1, comprising one or more additional bilayers, each comprising a layer of drug molecules and a polymer layer.

3. The system, according to claim 1, wherein the nanoparticle and one or more bilayer is encapsulated by a liposome.

4. The system, according to claim 1, comprising more than one bilayer, each with a different drug.

5. The system, according to claim 1, wherein the MNP comprises iron oxide.

6. The system, according to claim 1, wherein the drug and the polymer of a bilayer coating are oppositely charged.

7. The system, according to claim 1, wherein the polymer comprises a biodegradable material selected from alginate, heparin, chondroitin sulfate A & B, collagen, gelatin-A, dextran sulfate, chitosan, hyaluronic acid, poly-L-lysine, protamine sulfate, poly-L-arginine, carboxymethyl cellulose, polyglutamic acid, albumin, dextran amine, DNA and RNA.

8. The system, according to claim 1, comprising an antiretroviral drug selected from nucleoside reverse transcriptase inhibitors (NRTIs), non-nucleoside reverse transcriptase inhibitors (NNRTIs), nucleotide analog reverse transcriptase inhibitors (NtARTIs or NtRTIs), protease inhibitors (PIs), and integrase strand transfer inhibitors (INSTIs).

9. The system, according to claim 1, comprising an HIV latency-activating drug selected from protein kinase C (PKC) agonists, histone deacetylase (HDAC) inhibitors, Phaosphatase and tension homologs (PTENs) and nuclear factor kappa-light-chain-enhancer of activated B cells (NF-κB).

10. The system, according to claim 1, wherein the drug molecules are antagonists of a substance of abuse, wherein the antagonists are selected from cocaine antagonists, morphine antagonists, and methamphetamine antagonists.

11. A method of delivering a drug to a target location in a subject comprising administering to a subject in need of treatment the nanoparticle drug delivery system of claim 1; and localizing the nanoparticles to the target treatment area.

12. The method, according to claim 11, used to treat HIV.

13. The method, according to claim 11, wherein the localization of drug particles to the target treatment area is accomplished by applying an external non invasive magnetic force.

14. The method, according to claim 11, wherein the target treatment area comprises cellular structures with tight junctions.

15. The method, according to claim 14, wherein the target treatment area is the blood brain barrier and/or brain of a subject.

16. The method, according to claim 11, wherein the subject is human.

17. The method, according to claim 11, wherein the amount of nanoparticle drug delivery system present at a target treatment area is monitored by magnetic resonance imaging (MRI).

18. The method, according to claim 11, used to treat a disease selected from degenerative disorders, sensory and locomotor abnormalities, seizures, viral infections, immunological infections, mental and behavioral disorders, and localized central nervous system disease.

19. The method, according to claim 11, wherein the nanoparticle drug delivery system is used to treat HIV-associated neurocognitive disorders (HAND).

20. A nanoparticle drug delivery system, comprising a magnetic nanoparticle (MNP) encapsulated by two bilayer coatings, wherein the bilayer coating closest to the MNP comprises a layer of Tenofovir and a layer of dextran sulfate, and wherein the second bilayer comprises a layer of Vorinstat and a layer of dextran sulfate.
